(19) United States
(12) Patent Application Publication
(10) Pub. No.: US 2015/0152470 A1
(43) Pub. Date: Jun. 4, 2015

(54) THE USE OF ENDOTOXIN NEUTRALIZATION AS A BIOMARKER FOR INTESTINAL WALL DAMAGE

(71) Applicant: BioDtech, Inc., Birmingham, AL (US)

(72) Inventors: Michael G. Pepe, Birmingham, AL (US); Milton Keith Champion, Hoover, AL (US)

(21) Appl. No.: 14/406,117

(22) PCT Filed: Jun. 7, 2013
(86) PCT No.: PCT/US2013/044703

§ 371 Date: Dec. 5, 2014

Related U.S. Application Data
(60) Provisional application No. 61/657,219, filed on Jun. 8, 2012.

(51) Int. Cl. C12Q 1/37

(52) U.S. Cl. CPC C12Q 1/37; G01N 2400/50; G01N 2500/20; G01N 2500/04; G01N 2800/065

(57) ABSTRACT

Provided herein are methods for detecting endotoxin neutralization in a subject. Also provided are methods for determining the effectiveness of a therapeutic agent for treating intestinal wall damage. Further provided are methods of treating intestinal wall damage are also provided.

This application claims the benefit of U.S. Provisional Application No. 61/657,219, filed Jun. 8, 2012, which is hereby incorporated herein in its entirety.

### BACKGROUND

Mammalian survival is dependent on a rapid system to neutralize the potent immunostimulant effects of Gram negative bacterial endotoxin, a lipopolysaccharide found on the bacterial membrane. Endotoxin, is responsible for many, if not all, of the toxic effects that occur during Gram-negative bacterial sepsis. Chronic exposure to endotoxin is associated with disease states involving the gastrointestinal system. A standard approach to monitor the response to this exposure, or therapeutic agents to the exposure are nonexistent.

### SUMMARY

Methods for detecting neutralization of endotoxin are disclosed herein, for example, provided herein are methods for determining the effectiveness of a therapeutic agent for treating intestinal wall damage. For example, provided herein is a method of determining the effectiveness of a therapeutic agent for treating intestinal wall damage comprising a) administering a therapeutic agent to the subject; b) adding exogenous endotoxin to a first biological sample from the subject after treatment with the therapeutic agent; c) detecting exogenous endotoxin in the sample from step b); d) determining the amount of undetected exogenous endotoxin in the first sample by subtracting the detected exogenous endotoxin from step c) from the total exogenous endotoxin added in step b); and e) calculating the percentage of total endotoxin neutralization in the first sample, wherein the percentage of total endotoxin neutralization is the percentage of undetected exogenous endotoxin of the total exogenous endotoxin, and wherein a decrease in total endotoxin neutralization in the first sample as compared to control indicates that the therapeutic agent is effective for treating intestinal wall damage.

Further provided is a method of determining the effectiveness of a therapeutic agent for treating intestinal wall damage comprising a) administering a therapeutic agent to the subject; b) heating a first biological sample from the subject after treatment with the therapeutic agent; c) adding a selected amount of exogenous endotoxin to the first sample of step b); d) detecting exogenous endotoxin in the first sample from step c); e) determining the amount of undetected exogenous endotoxin in the first sample by subtracting the detected exogenous endotoxin from step d) from the total exogenous endotoxin added in step c); f) acidifying a second biological sample from a subject after treatment with the therapeutic agent; g) adding the selected amount of exogenous endotoxin to the second sample of step f); h) detecting exogenous endotoxin in the second sample from step g); i) determining the amount of undetected exogenous endotoxin in the second sample by subtracting the detected exogenous endotoxin from step h) from the total exogenous endotoxin added in step g); and j) calculating a percentage of protein endotoxin neutralization, utilizing the following equation:

((amount of undetected exogenous endotoxin the second sample−amount of undetected exogenous endotoxin in the first sample)/selected amount of exogenous endotoxin)×100;

wherein a decrease in the percentage of protein endotoxin neutralization after treatment as compared to a control indicates that the therapeutic agent is effective for treating intestinal wall damage.

Also provided is a method of treating intestinal wall damage in a subject comprising calculating levels of protein endotoxin neutralization, undetected exogenous endotoxin and enzymatic endotoxin neutralization in a biological sample from the subject; calculating an endotoxin neutralization ratio using the following formula:

(protein endotoxin neutralization+undetectable exogenous endotoxin)/enzymatic endotoxin neutralization;

a ratio of about 5 or greater indicating that the subject has intestinal wall damage; and administering a therapeutic agent for treating intestinal wall damage to the subject.

### BRIEF DESCRIPTION OF THE FIGURES

FIG. 1 is a graph showing total neutralization in male control and Crohn's disease samples.

FIG. 2 is a graph showing total neutralization in female control and Crohn's disease samples.

FIG. 3 is a graph showing enzymatic neutralization in male control and Crohn's disease samples.

FIG. 4 is a graph showing enzymatic neutralization in female control and Crohn's disease samples.

FIG. 5 is a graph showing protein neutralization in male control and Crohn's disease samples.

FIG. 6 is a graph showing protein neutralization in female control and Crohn's disease samples.

FIG. 7 is a graph showing undetectable endotoxin in male control and Crohn's disease samples.

FIG. 8 is a graph showing undetectable endotoxin in female control and Crohn's disease samples.

FIG. 9 is a graph showing neutralization ratio vs. age in female control samples.

FIG. 10 is a graph showing neutralization ratio vs. age in female Crohn's disease samples.

FIG. 11 is a graph showing neutralization ratio in male control and Crohn's disease samples.

FIG. 12 is a graph showing neutralization ratio in female control and Crohn's disease samples.

FIG. 13 is a graph showing neutralization ratio in male control and Crohn's disease samples.

FIG. 14 is a graph showing neutralization ratio in female control and Crohn's disease samples.

### DETAILED DESCRIPTION

Methods for determining endotoxin neutralization in biological samples are provided herein. For example, set forth herein is a method of determining the effectiveness of a therapeutic agent for treating intestinal wall damage comprising a) administering a therapeutic agent to the subject; b) adding exogenous endotoxin to a first biological sample from the subject after treatment with the therapeutic agent; c) detecting exogenous endotoxin in the sample from step b); d) determining the amount of undetected exogenous endotoxin in the first sample by subtracting the detected exogenous endotoxin from step c) from the total exogenous endotoxin added in step b); and e) calculating the percentage of total endotoxin neutralization in the first sample, wherein the percentage of total endotoxin neutralization is the percentage of undetected exogenous endotoxin of the total exogenous endotoxin, and wherein a decrease in total endotoxin neutralization in the first sample as compared to control indicates that the therapeutic agent is effective for treating intestinal wall damage.

In the methods set forth herein, intestinal wall damage can be any damage to the large intestine or the small intestine caused by a bacterial infection, a parasitic infection, a viral infection, radiation, a chemical, a drug, an inflammatory bowel disease (for example, Crohn's disease, ulcerative colitis, collagenous colitis, lymphocytic colitis, ischaemic colitis, diversion colitis, Behcet's disease and indeterminate colitis), celiac disease, intestinal cancer, colon cancer, intestinal obstruction, irritable bowel syndrome, an ulcer, or a perforation, to name a few.

As used throughout, by subject is meant an individual. Preferably, the subject is a mammal such as a primate, and, more preferably, a human. Non-human primates include marmosets, monkeys, chimpanzees, gorillas, orangutans, and gibbons, to name a few. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.).

As utilized throughout, the therapeutic agent for treating intestinal wall damage can be, but is not limited to, a chemical, a small or large molecule (organic or inorganic), a drug, a protein, a peptide, a cDNA, an antibody, an aptamer, a morpholino, a triple helix molecule, an siRNA, a shRNA, an miRNA, an antisense RNA, a ribozyme or any other compound now known or identified in the future that decreases intestinal wall damage. Agents for treating intestinal wall damage associated with inflammatory bowel diseases are known in the art. For example, aminosalicylates (for example, sulfasalazine or mesalamine), loperamide, antibiotics (for example, ciprofloxacin or metronidazole), corticosteroids (for example, budesonide or prednisone), immunomodulators (for example, azathioprine, mercaptopurine or cyclosporine), infliximab, adalimumab or combinations thereof can be utilized to treat Crohn's disease or ulcerative colitis.

Any appropriate route of administration may be employed to deliver the therapeutic agent. For example, parenteral, intravenous, subcutaneous, intramuscular, intraventricular, intracorporeal, intraperitoneal, rectal, or oral administration can be performed. Administration can be systemic or local. Therapeutic agents can be in a pharmaceutical composition that can be delivered locally to the area in need of treatment, for example by local injection or entubation. Multiple administrations and/or dosages can also be used.

After treatment with the therapeutic agent, a first biological sample is obtained from the subject. As used herein, a biological sample subjected to testing is a sample derived from a subject such as a mammal or human and includes, but is not limited to, any biological fluid, including a bodily fluid. Examples of bodily fluids include, but are not limited to, whole blood, plasma, serum, urine, saliva, ocular fluid, ascites, a stool sample, spinal fluid, tissue infiltrate, pleural effusions, lung lavage fluid, and the like. The biological fluid includes a cell culture medium or supernatant of cultured cells from the subject. For example, the sample can be a blood sample or a serum sample. The sample can also comprise a citrated or EDTA-containing sample.

A suitable time for obtaining the biological sample will vary depending on one or more factors, such as, but not limited to, the type of therapeutic agent, the extent of intestinal wall damage, the mode of administration, or whether single or multiple doses of the therapeutic agent must be administered to observe a therapeutic effect. The biological sample can be obtained at about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, 1 year or at any time in between, after administration of the therapeutic agent. In some cases, a control samples is collected from the subject prior to administration of the therapeutic agent. Such a control sample can be collected concurrently with administration of the therapeutic agent (so long as the agent has not had a biological effect on endotoxin) or 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, 1 year or at any time in between, before administration of the therapeutic agent.

Once a first biological sample is obtained from the subject, a selected amount of endogenous endotoxin is added to the sample. The selected amount of endogenous endotoxin can be added to achieve a concentration of about 50 EU/ml, 100 EU/ml, 150 EU/ml, 200 EU/ml, 250 EU/ml, 300 EU/ml, 350 EU/ml, 400 EU/ml, 450 EU/ml, 500 EU/ml, 550 EU/ml, 600 EU/ml, 650 EU/ml, 700 EU/ml, 750 EU/ml, 800 EU/ml, 850 EU/ml, 900 EU/ml, 950 EU/ml or 1000 EU/ml in the sample. The endotoxin can be obtained from commercial sources or prepared by various extraction methods that utilize chloroform, phenol, ether, acid and/or detergents. The endotoxin can also be prepared from a culture that is grown, heat-lysed and centrifuged to remove cell debris. For example, the endotoxin can be obtained from a Salmonella typhimurium LT2 stock

Prior to endotoxin detection, the methods set forth herein can further comprise heating the biological sample containing the selected amount of exogenous endotoxin; acidifying the heated sample to a pH of about 1 to 4; contacting the acidified sample with an acidic protease; and increasing the pH of the protease-treated sample to about 6 to 8. The acidic protease can be deactivated after contacting the sample with an active protease and before detecting endotoxin in the sample. Optionally, the acidic protease is inactivated by a pH of about 7.0 (e.g. 6.8-7.2).

As set forth above, prior to endotoxin detection, the sample can be heated to a temperature of about 55° C. to about 70° C. Therefore, the sample can be heated to a temperature of about 55° C., 56° C., 57° C., 58° C., 59° C., 60° C., 61° C., 62° C., 63° C., 64° C., 65° C., 66° C., 67° C., 68° C., 69° C. or about 70° C. The sample can be heated for about 20 to about 40 minutes. For example, the sample can be heated for about 40, 39, 38, 37, 36, 35, 34, 33, 32, 31, 30, 29, 28, 27, 26, 25, 24, 23, 22, 21 or about 20 minutes. The sample can be cooled after heating to about, for example, to about 18° C. to about 25° C. Therefore, the sample can be cooled to about 18° C., 19° C., 20° C., 21° C., 22° C., 23° C., 24° C. or to about 25° C.

The heated sample can then be acidified by adding an acid to the sample. For example, hydrochloric acid can be added to the sample to obtain a pH of about 1 to about 4. The acid can be at a normality or molarity sufficient to acidify a sample to a pH of about 1 to about 4 without unnecessary dilution of the sample. For example, the acid can be 1M HCl. Other acids include, but are not limited to, nitric acid, sulfuric acid and acetic acid. Optionally, an alkaline phosphatase inhibitor can be included when acidifying the sample or just prior to or after acidifying the sample.

The acidified sample can be contacted with an active acidic protease at a pH of less than 4, less than 3, less than 2 or about 1. Therefore, the pH can be about 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1 or about 1.0. Prior to acidifying the sample, the sample can be diluted to about 1 part sample to about 10 parts diluent. For example, the sample can be diluted to about 1 part sample to about 1 part diluent, to about 1 part sample to about 2 parts diluent, to about 1 part sample to about 3 parts diluent, to about 1 part sample to about 4 parts diluent, to about 1 part sample to about 5 parts diluent, to about 1 part sample to about 6 parts diluent, to about 1 part sample to about 7 parts diluent, to about 1 part sample to about 8 parts diluent, to about 1 part sample to about 9 parts diluent or to about 1 part sample to about 10 parts diluent. The diluent can be, but is not limited to, a buffer comprising divalent cations, for example, a Tris buffer comprising MgCl₂ or a Tris buffer comprising CaCl₂.

As used herein, acid, acidic, aspartic or aspartic acid proteases refer to proteases active at low pH. For example, the protease is active at a pH from about 0.0 to about 6.0 or any pH between 0.0 and 6.0, inclusive. Such proteases are inactive at a pH of about 6.0 to about 14.0. As used herein, an inactive acidic protease refers to a protease without proteolytic activity (i.e., a protease that is unable to cleave an amino acid sequence such as a polypeptide or protein). As used herein, an active acidic protease refers to a protease with proteolytic activity (i.e., a protease that is able to cleave an amino acid sequence). By way of example, an active acidic protease can be inactivated by a pH of 6.5 or higher (i.e., the protease is inactive in a solution with a pH of 6.5 or higher). The pH of a solution can be altered by addition of chemicals to the solution. For example, hydrochloric acid can be used to reduce pH and sodium hydroxide can be used to raise pH. As discussed above, pH adjustment is performed with an acidic or a basic solution of such normality or molarity to reduce unnecessary dilution of the sample. Phosphoric acid can be used to maintain a pH of about 6.5. Optionally, a pepsin inhibitor is used to inactivate pepsin. Pepsin inhibitors include, but are not limited to, acetamidine, N-acetyl-D-phenyalanyl-L-diiodotyrosine, N-acetyl-L-phenyalanyl-D-phenylalanine, p-aminobenzamidine, benzamidine, butyamine, diazoketones, ethylamine, pepstatin, and phenylactamidine.

Acid or acidic proteases, such as endopeptidases, are known and have been grouped into three families, namely, pepsin (A1), retropepsin (A2), and enzymes from pararetroviruses (A3). The members of families A1 and A2 are known to be related to each other, while those of family A3 show some relatedness to A1 and A2. Microbial acid proteases exhibit specificity against aromatic or bulky amino acid residues on both sides of the peptide bond, which is similar to pepsin, but their action is less stringent than that of pepsin. Acid proteases include microbial, fungal, viral, animal and plant acidic proteases. Microbial aspartic proteases can be broadly divided into two groups, (i) pepsin-like enzymes produced by Aspergillus, Penicillium, Rhizopus, and Neurospora and (ii) rennin-like enzymes produced by Endothia and Mucor spp (Rao et al., Microbiology and Molecular Biology 62(3):597-635 (1998); Richter et al., Biochem. J. 335:481-90 (1998)). Examples of acidic proteases include, but are not limited to, pepsins, including pepsins A, B and C; rennin; chymosin; plasmepsin; cathepsins, such as, for example, cathepsin D and cathepsin E; human urinary acid protease; and viral proteases like HIV protease. Fungal proteases include, but are not limited to, fungal proteases derived from Neurospora oryzae, Mucor pusillus, Mucor miehei, Aspergillus niger, Rhizopus chinensis, or Endothia parasitica. Microbial proteases include, but are not limited to, yeast proteinase A, aspergillopepsinogen, rhizopuspepsin, penicillopepsin, and endothiapepsin.

In the methods set forth herein, the pH of a protease-treated sample can be increased to a pH greater than about 6, greater than about 6.5, greater than about 7, greater than about 7.5 or about 8 by addition of a base to the protease-treated sample. Therefore, the pH can be about 6, 6.1, 6.2, 6.3, 6.4, 6.5, 6.6, 6.7, 6.8, 6.9, 7.0, 7.1, 7.2, 7.3, 7.4, 7.5, 7.5, 7.7, 7.8, 7.9 or about 8. The base can be at a normality or molarity that the pH adjustment is made without unnecessary dilution of the sample. For example, 0.5 N sodium hydroxide can be used to increase pH. Other examples of bases include, but are not limited to, potassium hydroxide and ammonia. Prior to increasing the pH of the sample, from about 6 to about 8, the sample can be diluted to about 1 part sample to about 10 parts diluent. For example, the sample can be diluted to about 1 part sample to about 1 part diluent, to about 1 part sample to about 2 parts diluent, to about 1 part sample to about 3 parts diluent, to about 1 part sample to about 4 parts diluent, to about 1 part sample to about 5 parts diluent, to about 1 part sample to about 6 parts diluent, to about 1 part sample to about 7 parts diluent, to about 1 part sample to about 8 parts diluent, to about 1 part sample to about 9 parts diluent or to about 1 part sample to about 10 parts diluent. The diluent can be, but is not limited to, a buffer comprising divalent cations, for example a Tris buffer comprising MgCl₂ or a Tris buffer comprising CaCl₂.

In the methods set forth herein, endotoxin can be detected via methods standard in the art, for example, and, not to be limiting, these include gel-clot assays, turbidimetric assays, and chromogenic assays. The PyroGene® Recombinant Factor C Endotoxin detection System (Lonza 50-658U; Allendale, N.J.) is an example of a fluorescence assay that can be utilized.

Once the amount of exogenous endotoxin is detected in the first biological sample, the amount of undetected exogenous endotoxin is calculated by subtracting the amount of exogenous endotoxin detected in the first biological sample from the total exogenous endotoxin added to the first biological sample. After the amount of undetected exogenous endotoxin is calculated, the percentage of total endotoxin neutralization in the first sample is calculated. The percentage of total endotoxin neutralization is the percentage of undetected exogenous endotoxin of the total exogenous endotoxin added to the first biological sample (amount of undetected exogenous endotoxin/total exogenous endotoxin)×100. A decrease in total endotoxin neutralization in the first sample as compared to control indicates that the therapeutic agent is effective for treating intestinal wall damage. The control can be the percentage of total endotoxin neutralization in a sample from the same subject prior to or at about the same time as administration of the therapeutic agent, the percentage total endotoxin neutralization in a sample from the same subject after administration of a different therapeutic agent or the percentage of total endotoxin neutralization in a reference sample. An increase in total endotoxin neutralization as compared to control indicates that the therapeutic agent is not effective in treating intestinal wall damage. If an increase in total endotoxin neutralization is observed after treatment with a therapeutic agent, one of skill in the art can, for example, discontinue treatment, alter the dosage of the therapeutic agent or administer a different therapeutic agent.

As utilized throughout, the reference sample can be from the same subject or a different subject before or at about the same time as administration of the therapeutic agent. The reference sample can also be a sample obtained from a subject after the effects of the therapeutic agent have subsided. The reference sample can also be from a healthy subject. This method of determining the effectiveness of a therapeutic agent for treating intestinal wall damage can further comprise f) heating a second biological sample from the subject after treatment with the therapeutic agent; g) adding exogenous endotoxin to the second sample of step f); h) detecting exogenous endotoxin in the second sample from step g); i) determining the amount of undetected exogenous endotoxin by subtracting the detected exogenous endotoxin from step h) from the amount of added exogenous endotoxin from step g); and j) calculating the percentage of enzymatic endotoxin neutralization utilizing the following equation:

((amount of undetected exogenous endotoxin in the second sample from step h−amount of undetected exogenous endotoxin in the first sample from step b)/amount of total exogenous endotoxin added in step g)×100;

wherein an increase in the percentage of enzymatic endotoxin neutralization in the second biological sample as compared to control indicates that the therapeutic agent is effective for treating intestinal wall damage. In this method, the second biological sample can be heated to from about 55° C. to about 70° C. Also, in this method, the same amount of exogenous endotoxin is added to the first and the second biological sample.

As utilized throughout, the percentage of enzymatic endotoxin neutralization is the percentage of endotoxin that is not detected due to heat inactivation of enzymatic processes in the biological sample, for example, in blood plasma. The control can be the percentage of enzymatic endotoxin neutralization in a sample from the same subject prior to or at about the same time as administration of the therapeutic agent, the percentage total enzymatic endotoxin neutralization in a sample from the same subject after administration of a different therapeutic agent or the percentage of total enzymatic endotoxin neutralization in a reference sample. A decrease in enzymatic endotoxin neutralization as compared to control indicates that the therapeutic agent is not effective in treating intestinal wall damage. If a decrease in enzymatic endotoxin neutralization is observed after treatment with a therapeutic agent, one of skill in the art can, for example, discontinue treatment, alter the dosage of the therapeutic agent or administer a different therapeutic agent.

Further provided is a method of determining the effectiveness of a therapeutic agent for treating intestinal wall damage comprising a) administering a therapeutic agent to the subject; b) heating a first biological sample from the subject after treatment with the therapeutic agent; c) adding a selected amount of exogenous endotoxin to the first sample of step b); d) detecting exogenous endotoxin in the first sample from step c); e) determining the amount of undetected exogenous endotoxin in the first sample by subtracting the detected exogenous endotoxin from step d) from the total exogenous endotoxin added in step c); f) acidifying a second biological sample from a subject after treatment with the therapeutic agent; g) adding the selected amount of exogenous endotoxin to the second sample of step f); h) detecting exogenous endotoxin in the second sample from step g); i) determining the amount of undetected exogenous endotoxin in the second sample by subtracting the detected exogenous endotoxin from step h) from the total exogenous endotoxin added in step g); and j) calculating a percentage of protein endotoxin neutralization, utilizing the following equation:

((amount of undetected exogenous endotoxin the second sample−amount of undetected exogenous endotoxin in the first sample)/selected amount of exogenous endotoxin)×100;

wherein a decrease in the percentage of protein endotoxin neutralization after treatment as compared to a control indicates that the therapeutic agent is effective for treating intestinal wall damage. In this method, the first biological sample can be heated to from about 55° C. to about 70° C. Also, in this method, the second biological sample can be acidified to a pH of less than 4, less than 3, less than 2 or about 1. Further, in this method, the same amount of exogenous endotoxin is added to the first and the second biological sample.

As utilized throughout, the percentage of protein endotoxin neutralization is the percentage of exogenous endotoxin not detected due to acid-inactivation via binding of blood plasma proteins, including immunoglobulins. Acid-inactivation is utilized to denature proteins in the biological sample.

Further provided is a method of treating intestinal wall damage in a subject comprising calculating levels of protein endotoxin neutralization, undetected exogenous endotoxin and enzymatic endotoxin neutralization in a biological sample from the subject; and calculating an endotoxin neutralization ratio using the following formula:

(protein endotoxin neutralization+undetectable exogenous endotoxin)/enzymatic endotoxin neutralization);

a ratio of about 5 or greater indicating that the subject has intestinal wall damage; and administering a therapeutic agent for treating intestinal wall damage to the subject. This ratio is known as a neutralization ratio and is roughly equivalent to the ratio of protein inactivation to enzymatic inactivation. This ratio can also be utilized to determine the severity of the disease as lower ratios will correspond to moderate intestinal wall damage and higher ratios will correspond to more severe intestinal wall damage. For example, and not to be limiting, if the intestinal wall damage is associated with Crohn's disease, lower ratios correspond to moderate cases of Crohn's disease that can be treated with an immunomodulator, an anti-inflammatory and/or an antibiotic. Higher ratios correspond to more severe cases of Crohn's disease that may not respond to an immunomodulator, an anti-inflammatory and/or an antibiotic. Therefore, one of skill in the art would know to administer a therapeutic agent for more severe cases of Crohn's disease, such as a corticosteroid or infliximab. The neutralization ratio can also be used to determine the effectiveness of a therapeutic agent in treating intestinal wall damage. If there is a decrease in the neutralization ratio in a sample from the subject after administration of a therapeutic agent, as compared to control, this indicates that the therapeutic agent is effective for treating intestinal wall damage. The control can be a sample from the subject prior to administration of the therapeutic agent or a reference sample.

In this method, protein endotoxin neutralization is calculated by a) heating a first biological sample from the subject; b) adding a selected amount of exogenous endotoxin to the first sample of step a); c) detecting exogenous endotoxin in the first sample from step b); d) determining the percentage of undetected exogenous endotoxin in the first sample using the following equation:

((total exogenous endotoxin added in step b−the detected exogenous endotoxin from step c)/the total exogenous endotoxin added in step b)×100;

e) acidifying a second biological sample from a subject; f) adding the selected amount of exogenous endotoxin to the second sample of step e); g) detecting exogenous endotoxin in the second sample from step f); h) determining the percentage of undetected exogenous endotoxin after acidification using the following equation:

((total exogenous endotoxin added in step f−the detected exogenous endotoxin from step g)/the total exogenous endotoxin added in step f)×100;

and
i) calculating protein endotoxin neutralization, utilizing the following equation:

percentage of undetected exogenous endotoxin in step h)−percentage of undetected exogenous endotoxin in step d).

In this method, undetectable endotoxin is the percentage of exogenous endotoxin that is not detected even after heat and/or acid inactivation. Undetectable endotoxin is calculated by a) acidifying a biological sample from a subject; b) adding a selected amount of exogenous endotoxin to the sample of step a); c) detecting exogenous endotoxin in the sample from step b); and d) determining the percentage of undetectable exogenous endotoxin after using the following equation:

((total exogenous endotoxin added in step b−the detected exogenous endotoxin from step c)/the total exogenous endotoxin added in step b)×100.

In this method, enzymatic endotoxin neutralization is calculated by a) adding exogenous endotoxin to a first biological sample from the subject; b) detecting exogenous endotoxin in the first sample from step a); c) determining the amount of undetected exogenous endotoxin in the first sample by subtracting the detected exogenous endotoxin from step b) from the total exogenous endotoxin added in step a); d) calculating the percentage of undetected exogenous endotoxin in the first sample using the following equation:

(the amount of undetected exogenous endotoxin in step c/total exogenous endotoxin added in step a)×100;

e) heating a second biological sample from the subject; f) adding exogenous endotoxin to the second sample of step e); g) detecting exogenous endotoxin in the second sample from step f); h) determining the amount of undetected exogenous endotoxin in the second sample by subtracting the detected exogenous endotoxin from step g) from the amount of added exogenous endotoxin from step f); i) calculating the percentage of undetected exogenous endotoxin in the second sample using the following equation:

(the amount of undetected exogenous endotoxin in step h/total exogenous endotoxin added in step f)×100;

and

j) calculating the percentage of enzymatic endotoxin neutralization utilizing the following equation:

((percentage of undetected exogenous endotoxin in the second sample−percentage of undetected exogenous endotoxin in the first sample)/amount of total exogenous endotoxin added in step a)×100.

If a subject has a neutralization ratio of about 5 or greater, a therapeutic agent can be administered to treat intestinal wall damage. The ratio can be about 5, 5.1, 5.2, 5.3, 5.4, 5.5, 5.6, 5.7, 5.8, 5.9, 6.0, 6.5, 7.0, 7.5, 8.0, 8.5, 9.0, 9.5, 10, 15, 20, 25, 30, 35, 40, 45 or greater. As set forth above, the therapeutic agent can be, but is not limited to, a chemical, a small or large molecule (organic or inorganic), a drug, a protein, a peptide, a cDNA, an antibody, an aptamer, a morpholino, a triple helix molecule, an siRNA, a shRNA, an miRNA, an antisense RNA, a ribozyme or any other compound now known or identified in the future that decreases intestinal wall damage.

By treat, treating, or treatment is meant a method of reducing or slowing intestinal wall damage. Treatment can also refer to a method of reducing the disease or condition associated with intestinal wall damage or reducing or slowing one or more of the symptoms. The treatment or slowing can be any reduction or slowing from native levels and can be, but is not limited to, the complete ablation of the disease or the symptoms of the disease. Treatment can range from a positive change in a symptom or symptoms to complete amelioration as detected by art-known techniques. For example, a disclosed method is considered to be a treatment if there is about a 10% reduction in intestinal wall damage in a subject when compared to native levels in the same subject or control subjects or a 10% increase in weight gain. Thus, the reduction or improvement can be about a 10, 20, 30, 40, 50, 60, 70, 80, 90, 100%, or any amount of reduction or improvement in between as compared to native or control levels.

The choice of therapeutic agent will depend on the symptoms and medical history of the subject. One of skill in the art can administer one or more therapeutic agents suitable for treating intestinal wall damage, depending on the severity or the stage of a disease, as determined by the neutralization ratio. For example, if the subject shows signs or symptoms of inflammatory bowel disease, one of skill in the art can administer aminosalicylates (for example, sulfasalazine or mesalamine), loperamide, antibiotics (for example, ciprofloxacin or metronidazole), corticosteroids (for example, budesonide or prednisone), immunomodulators (for example, azathioprine, mercaptopurine or cyclosporine), infliximab, adalimumab or combinations thereof to treat the inflammatory bowel disease. In another example, if the subject exhibits signs or symptoms of viral, bacterial or parasitic infection, an appropriate antiviral, antibacterial or antiparasitic agent can be administered.

Antibacterial agents include, but are not limited to, antibiotics (for example, penicillin and ampicillin), sulfa drugs and folic acid analogs, Beta-lactams, aminoglycosides, tetracyclines, macrolides, lincosamides, streptogramins, fluoroquinolones, rifampin, mupirocin, cycloserine, aminocyclitol and oxazolidinones.

Antiparasitic agents include, but are not limited to, antihelmintics, antinematodal agents, antiplatyhelmintic agents, antiprotozoal agents, amebicides, antimalarials, antitrichomonal agents, aoccidiostats and trypanocidal agents.

Any of the therapeutic agents set forth herein can be combined with chemotherapy, immunotherapy, anti-inflammatory agents, radiation or surgery.

The agents described herein can be provided in a pharmaceutical composition. Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the agent described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected agent without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia Pa., 2005. Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.).

Compositions containing the agent(s) described herein suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Administration can be carried out using therapeutically effective amounts of the agents described herein for periods of time effective to treat intestinal wall damage. The effective amount may be determined by one of ordinary skill in the art and includes exemplary dosage amounts for a mammal of from about 0.5 to about 200 mg/kg of body weight of active compound per day, which may be administered in a single dose or in the form of individual divided doses, such as from 1 to 4 times per day. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about 5 mg/kg of body weight of active compound per day.

According to the methods taught herein, the subject is administered an effective amount of the agent. The terms effective amount and effective dosage are used interchangeably. The term effective amount is defined as any amount necessary to produce a desired physiologic response. Effective amounts and schedules for administering the agent may be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the activity of the specific compound employed, the metabolic stability and length of action of that compound, the species, age, body weight, general health, sex and diet of the subject, the mode and time of administration, rate of excretion, drug combination, and severity of the particular condition and can be determined by one of skill in the art. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products.

Any appropriate route of administration may be employed, for example, parenteral, intravenous, subcutaneous, intramuscular, intraventricular, intracorporeal, intraperitoneal, rectal, or oral administration. Administration can be systemic or local. Pharmaceutical compositions can be delivered locally to the area in need of treatment, for example by topical application or local injection. Multiple administrations and/or dosages can also be used. Effective doses can be extrapolated from dose-response curves derived from in vitro or animal model test systems.

In an example in which a nucleic acid is employed, such as an antisense or an siRNA molecule, the nucleic acid can be delivered intracellularly (for example by expression from a nucleic acid vector or by receptor-mediated mechanisms), or by an appropriate nucleic acid expression vector which is administered so that it becomes intracellular, for example by use of a retroviral vector (see U.S. Pat. No. 4,980,286), or by direct injection, or by use of microparticle bombardment (such as a gene gun; Biolistic, Dupont), or coating with lipids or cell-surface receptors or transfecting agents, or by administering it in linkage to a homeobox-like peptide which is known to enter the nucleus (for example Joliot et al., Proc. Natl. Acad. Sci. USA 1991, 88:1864-8). siRNA carriers also include, polyethylene glycol (PEG), PEG-liposomes, branched carriers composed of histidine and lysine (HK polymers), chitosan-thiamine pyrophosphate carriers, surfactants (for example, Survanta and Infasurf), nanochitosan carriers, and D5W solution. The present disclosure includes all forms of nucleic acid delivery, including synthetic oligos, naked DNA, plasmid and viral delivery, whether integrated into the genome or not.

Physical transduction techniques can also be used, such as liposome delivery and receptor-mediated and other endocytosis mechanisms (see, for example, Schwartzenberger et al., Blood 87:472-478, 1996) to name a few examples. These methods can be used in conjunction with any of these or other commonly used gene transfer methods.

All of the calculations set forth herein can be performed by a computer. For example, a computer-readable medium, on which are stored executable instructions that, when executed by a computer processor, perform any of the methods or calculations set forth herein is provided. In another example, provided herein is a computer system comprising software for effecting the following steps: a) receiving a set of detected exogenous endotoxin values for at least one sample from a subject after treatment with a therapeutic agent for treating intestinal wall damage; b) determining the amount of undetected exogenous endotoxin in the sample; and c) calculating the percentage of total endotoxin neutralization in the sample; wherein a decrease in total endotoxin neutralization in the first sample as compared to control indicates that the therapeutic agent is effective for treating intestinal wall damage.

Also provided is a computer system comprising software for effecting the following steps: a) receiving a set of detected exogenous endotoxin values for at least one sample from a subject after treatment with a therapeutic agent for treating intestinal wall damage, wherein the sample is heated; b) determining the amount of undetected exogenous endotoxin in the heated sample; and c) calculating the percentage of enzymatic endotoxin neutralization in the sample; wherein an increase in the percentage of enzymatic endotoxin neutralization in the first sample as compared to control indicates that the therapeutic agent is effective for treating intestinal wall damage.

Further provided is a computer system comprising software for effecting the following steps: a) receiving a set of detected exogenous endotoxin values for at least one first sample from a subject after treatment with a therapeutic agent for treating intestinal wall damage, wherein the sample is heated; b) receiving a set of detected exogenous endotoxin values for at least one second sample from a subject after treatment with a therapeutic agent for treating intestinal wall damage, wherein the sample is acidified; c) determining the amount of undetected exogenous endotoxin in the heated sample; d) determining the amount of undetected exogenous endotoxin in the acidified sample; and e) calculating the percentage of protein endotoxin neutralization in the sample; wherein a decrease in the percentage of enzymatic endotoxin neutralization in the first sample as compared to control indicates that the therapeutic agent is effective for treating intestinal wall damage.

Also provided is a computer system comprising software for effecting the following steps: a) receiving a set of protein endotoxin neutralization values, undetected exogenous endotoxin values and enzymatic endotoxin neutralization values from at least one biological sample from a subject; and b) calculating an endotoxin neutralization ratio, wherein a ratio of about 5 or greater indicates that the subject has intestinal wall damage. The computer system can further comprise a processor, configured to execute instructions and memory on which are stored executable instructions, wherein the instructions are configured to perform any of the methods or calculations set forth herein.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

A number of embodiments have been described. Nevertheless, it will be understood that various modifications may be made. Accordingly, other embodiments are within the scope of the following claims.

### Examples

In this study, the extent and nature of endotoxin neutralization in blood plasma samples was determined in order to develop a system for using endotoxin as a biomarker for Crohn's Disease (CD). The data show that both male and female patients with CD have a decreased capacity of enzymatic endotoxin neutralization and an increased capacity of endotoxin neutralization via protein binding. In addition, it was found that CD males and both control and CD females have an increased population of undetectable endotoxin when compared to control males. These individual results were combined into a formula that allows assignment of an Endotoxin Neutralization Ratio to each patient sample. This ratio gave 95% accuracy in determining an intestinal disease state in male patients, 85% in female patients and 88% for all patients. In addition, this ratio indicated an age dependence in female CD patients as well as disease severity and/or medication status dependence in both male and female CD patients. Lastly, data is given for similar application in Ulcerative Colitis (UC) patients. Here, the Endotoxin Neutralization Ratio gave a 75% accuracy in males, 83% in females and 80% in all patients.

### Protocols

### ESP—Endotoxin Sample Preparation Protocol

1. Pipette citrated plasma into glass tube and cover with parafilm.
2. Heat tube in 55-60° C. water bath for 25 minutes.
3. Mix 30 μl of heat-inactivated citrated plasma with 270 μl ESP Buffer #1.
a. ESP Buffer #1-10 mM Tris-HCl pH 1.5
b. This step lowers the sample pH which prepares the sample for ESP enzymatic digestion. In addition, the pH shift further inactivates blood plasma enzymes.

### 4. Add 30 μl of ESP Protease Solution.

a. ESP Protease Solution—5% Pepsin in 10 mM Tris-HCl pH 1.5
   5. Incubate tube in a 37° C. shaking water bath for 120 minutes.
   6. Mix 50 μl sample with 450 μl ESP Buffer #2.
a. ESP Buffer #2-10 mM Tris-HCl pH 8.5
b. This step neutralizes the sample for recombinant Factor C testing.
   7. Test each sample with and without PPC in the Lonza PyroGene® assay (Allentown, N.J.).

### Endotoxin Neutralization Protocol

Each sample is tested after 4 treatments:

### Endogenous

1. Citrated plasma is treated with the ESP™ protocol and tested with the Lonza PyroGene® assay.

### Spiked

1. 90 μl citrated plasma is mixed with 10 μl endotoxin-free water.
2. 72 μl of this is mixed with 8 μl endotoxin stock solution.
3. Treat with the ESP™ protocol and test with the Lonza PyroGene® assay.

### Heated

1. 90 μl citrated plasma is mixed with 10 μl endotoxin-free water.
2. Sample is heated in a 55-60° C. water bath for 25 minutes.
3. 72 μl of this is mixed with 8 μl endotoxin stock solution.
4. Treat with the ESP™ protocol and test with the Lonza PyroGene® assay.

### Acidified

1. 90 μl citrated plasma is mixed with 10 μl 2 M hydrochloric acid.
2. 72 μl of this is mixed with 8 μl endotoxin stock solution.
3. Treat with the ESP™ protocol and test with the Lonza PyroGene® assay.

### Results

### Rapid Neutralization Data

In development of the ESP assay, samples with excellent positive product control (PPC) values but poor recovery of spiked endotoxin were common. This was due to a rapid neutralization of endotoxin upon addition to intact blood plasma. When an extracted, purified endotoxin stock was added to plasma only 0.5-0.7% was recovered after 10 minutes. If the pH was lowered prior to endotoxin addition the results were similar (0-1.3% recovery). However, if the pH was lowered and the plasma was heat-inactivated in a 60° C. water bath for 30 minutes 50.1-62.4% could be recovered. Next, neutralization was compared to a crude endotoxin lysate prepared with gentle heat-lysis and centrifugation from a Salmonella typhimurium LT2 stock. After 5 minutes in intact plasma the purified endotoxin was 99.4% neutralized compared to 76.2% for the crude stock. Performing a time-course experiment it was found that almost all the neutralization happened within the first 5 minutes of incubation and there was only an additional 5-10% of neutralization of the crude endotoxin if the samples were allowed to incubate for up to 120 minutes.

### Total Neutralization

Given the rapid neutralization of endotoxin in plasma, circulating endotoxin is a poor biomarker for any condition. However, it is possible that the level of endotoxin neutralization can indicate the level of immune system activation. To test this, 30 citrated plasma samples were collected from normal, human adults (10 male, 20 female). After ensuring that the samples contained no circulating endotoxin, each sample was spiked with a known amount of a crude endotoxin stock solution, allowed to incubate at room temperature, treated with the ESP treatment protocol and the remaining endotoxin concentration was measured with a recombinant Factor C assay. The percentage of endotoxin neutralized was determined and termed total endotoxin neutralization (TEN). A significant difference in TEN between male and female patients was observed. The average male had a TEN of 74.6% with a range from 63.0 to 82.9%. The average female had a TEN of 86.7% with a range from 81.9 to 94.1%. The difference between the sexes was 12.1%, was statistically significant (p=4.8700e-7) and contained only one sample that overlapped the two populations. In addition, there was an age component to TEN in males but not females. The slope of a linear regression line fitted to the male data had a value of 0.6821, indicating that TEN increases with age. However, the same regression line fitted to the female data had a value of only −0.0380, indicating no significant change with age. We interpret these results to indicate that females have a more robust reaction to endotoxin than males. This is most likely due to a higher susceptibility of infection in females. This is supported by the data that, as males age, they become more similar to females in their TEN value, most likely as a result of exposure to bacterial infections as they age.

### Crohn's Disease

A total of 60 patient samples were tested:
10—male controls
10—males with Crohn's Disease
20—female controls 20—females with Crohn's Disease

Each patient sample was tested in triplicate with each of the 4 neutralization protocols with and without PPC controls according to the ESP™ protocol. Endotoxin was measured using the Lonza PyroGene® assay according to manufacturer's specifications. Data given are the average of 3 tests.

### Total Endotoxin Neutralization

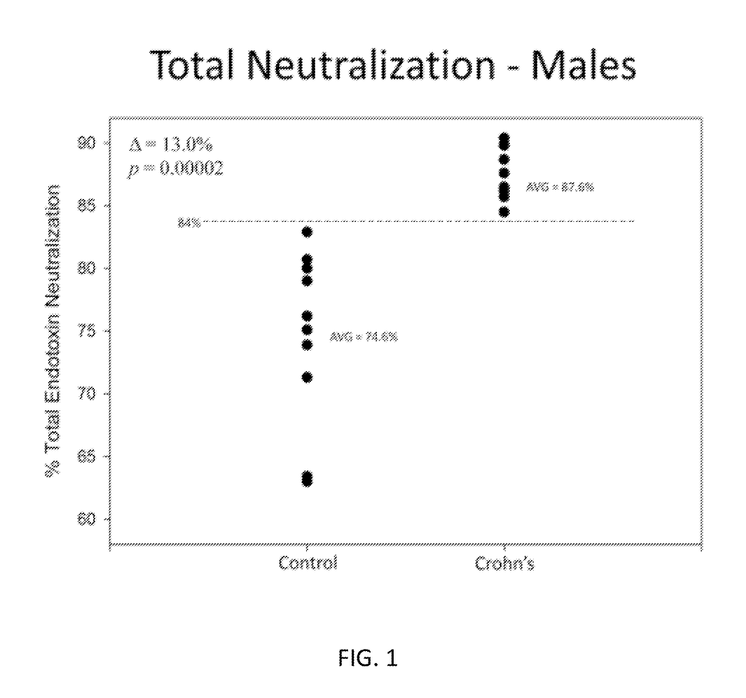

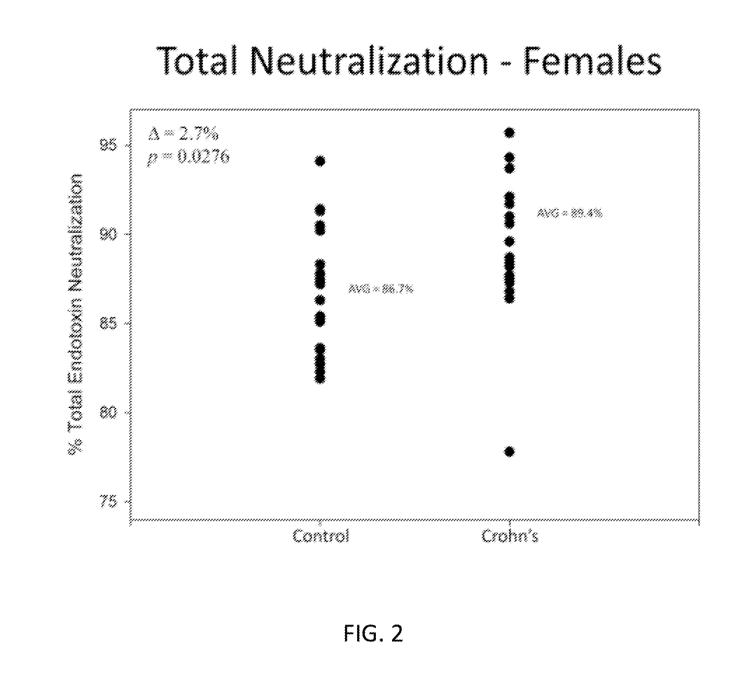

There was a significant difference in Total Endotoxin Neutralization between males and females and between male control and CD patients. In males, Total Endotoxin Neutralization was 74.6% compared to 87.6% for CD samples (p=0.00002). The separation in the males was clear enough that a line at 84% on the y-axis could demarcate control from CD samples with 100% accuracy (FIG. 1). In control females, Total Endotoxin Neutralization was almost identical to CD males (86.7% vs. 87.6%, p=0.5000). The level in females went up slightly in CD patients (89.4%) (FIG. 2). These results suggest that Total Endotoxin Neutralization can be a marker for CD in males.

### Enzymatic Endotoxin Neutralization

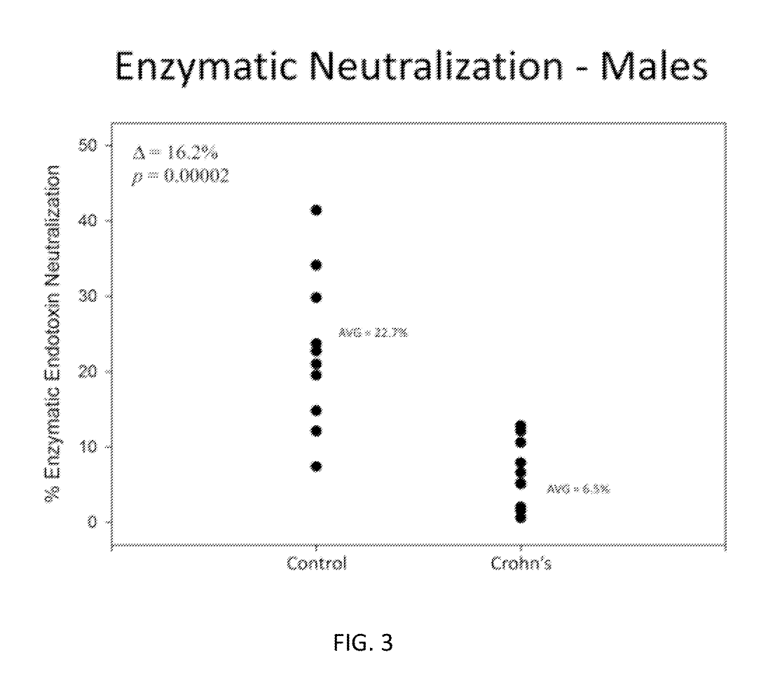

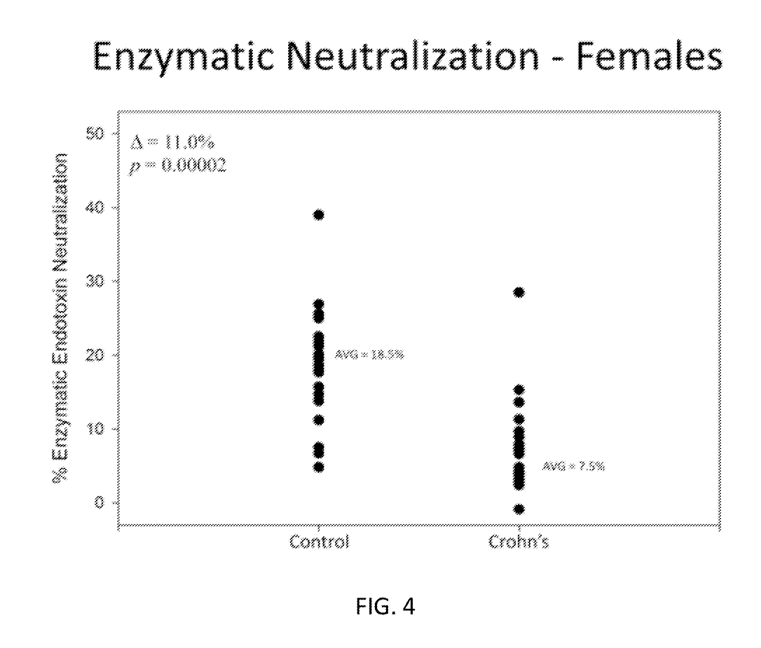

The Enzymatic Endotoxin Neutralization was very similar for males and females but was significantly different in the CD samples as compared to the controls. Control males had an average Enzymatic Endotoxin Neutralization of 22.7% that decreased to 6.5% in the CD samples (p=0.00002) (FIG. 3). Similarly, the average female control was 18.5% and decreased to 7.5% in CD (p=0.00002) (FIG. 4). This shows that Enzymatic Endotoxin Neutralization can be useful in defining neutralization as a biomarker in CD.

### Protein Endotoxin Neutralization

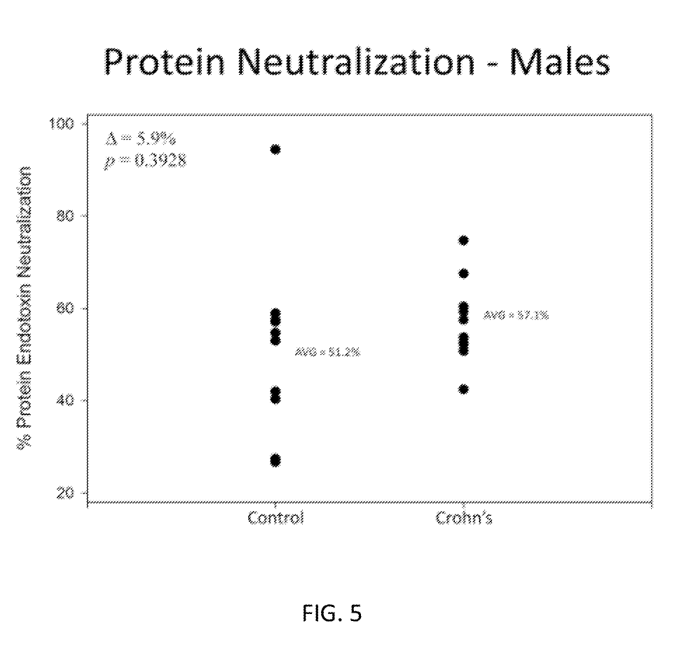

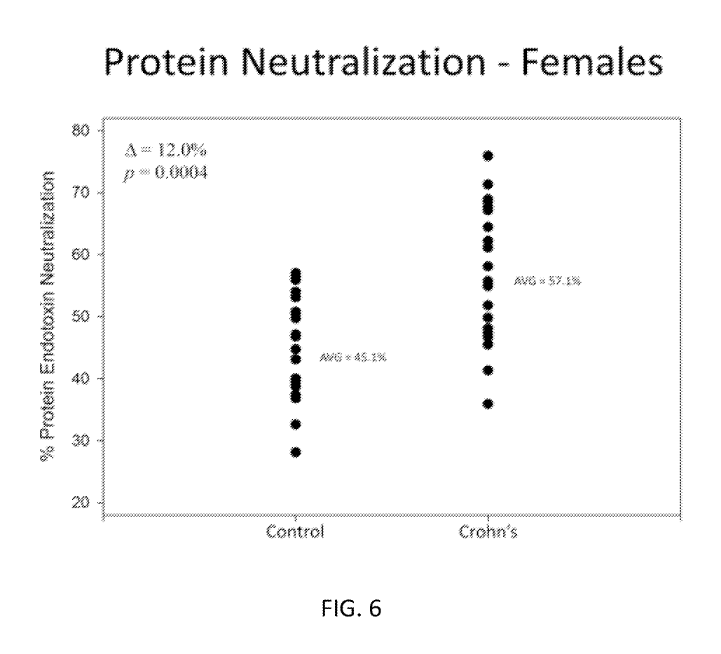

Much like enzymatic neutralization, the Protein Endotoxin Neutralization was comparable between sexes but increased with CD. Control males and females had an average of 51.2% and 45.1%, respectively, and each was increased to 57.1% in CD (FIGS. 5 and 6). In the males this was not a significant increase (p=0.3928) due largely to an outlier at 94%. Omitting this sample makes the average 46.4%, much closer to the female controls, with a p=0.0486. In females there was a 12% difference between the controls and CD samples (p=0.0004). These results suggest that Protein Endotoxin Neutralization can be a valuable component in defining an endotoxin neutralization biomarker for CD.

### Undetectable Endotoxin

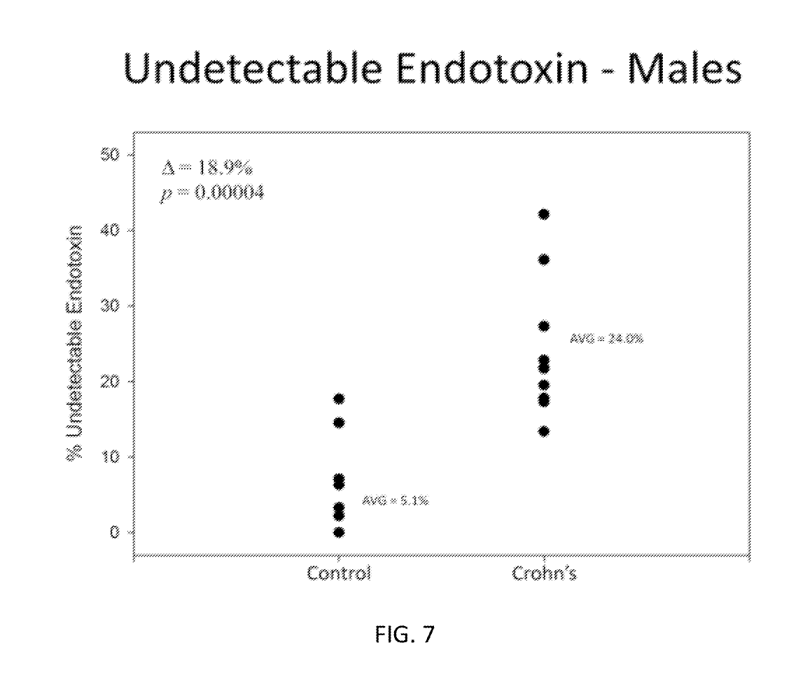

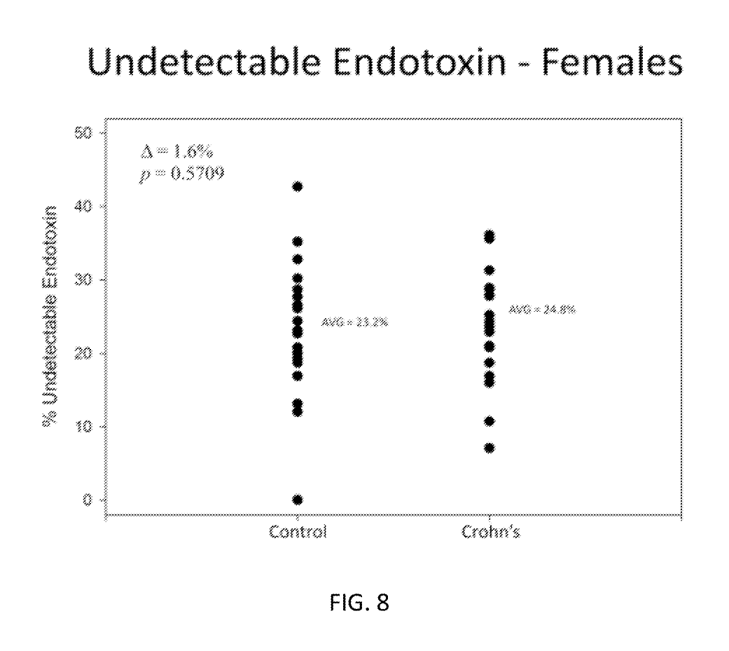

The Undetectable Endotoxin closely mirrored Total Endotoxin Neutralization in that all females and CD males had similar values while the control males were significantly lower. The male controls contained an average of only 5.1% Undetectable Endotoxin. This increased to 24.0% in the CD group (p=0.00004), very similar to both the female control and female CD samples where there was negligible difference (FIGS. 7 and 8).

Summarily, these results show that the Total Endotoxin Neutralization increased in males with CD. This increase was approximately 13%. Internal to this number was a 16% decrease in Enzymatic Endotoxin Neutralization, a 6% increase in Protein Endotoxin Neutralization and a 19% increase in Undetectable Endotoxin.

In females there was little difference in the Total Endotoxin Neutralization. However, the internals were similar to the males. The 3% difference is explained by an 11% decrease in Enzymatic Endotoxin Neutralization, a 12% increase in Protein Endotoxin Neutralization and a 2% increase in Undetectable Endotoxin.

The major difference between the sexes in endotoxin neutralization was the amount of Undetectable Endotoxin in the control groups (19% in males, 2% in females). Since the enzymatic and protein neutralization components roughly cancel each other out, it is possible that this is responsible for the difference in Total Endotoxin Neutralization. Due to an increased susceptibility of infection, it is likely that females (both control and CD) have high-affinity proteins, such as immunoglobulins, that bind and neutralize endotoxin and can withstand both heat- and acid-inactivation. This high-affinity molecule is only present in males with CD where infection exposure is increased to the level of females.

Set forth below is the formula that was developed to measure the ratio of enzymatic neutralization to protein neutralization. Since Undetectable Endotoxin is likely caused by an unknown protein, it was included in protein neutralization.

(protein endotoxin neutralization+undetectable exogenous endotoxin)/enzymatic endotoxin neutralization).

### Endotoxin Neutralization Ratio

The control male Endotoxin Neutralization Ratio samples clustered in an area between 0.85 to 5.11, with one exception at 12.76. The male CD samples were much more diverse. There was a cluster of 4 patients, between 5-10, a cluster of 3 patients between 10-20 and individual points at 42.25, 55.50 and 146.83. The 7 patients with the lowest ratios all had moderate cases of CD and were prescribed either an immunomodulator or antibiotic. The patient at 42.25 had a mild case of CD but was not prescribed either drug type (though he was prescribed an anti-inflammatory). The patients at 55.50 and 146.83 both had severe cases of CD and were not prescribed an immunomodulator, antibiotic or anti-inflammatory. There does not appear to be an age factor in the Endotoxin Neutralization Ratio in males.

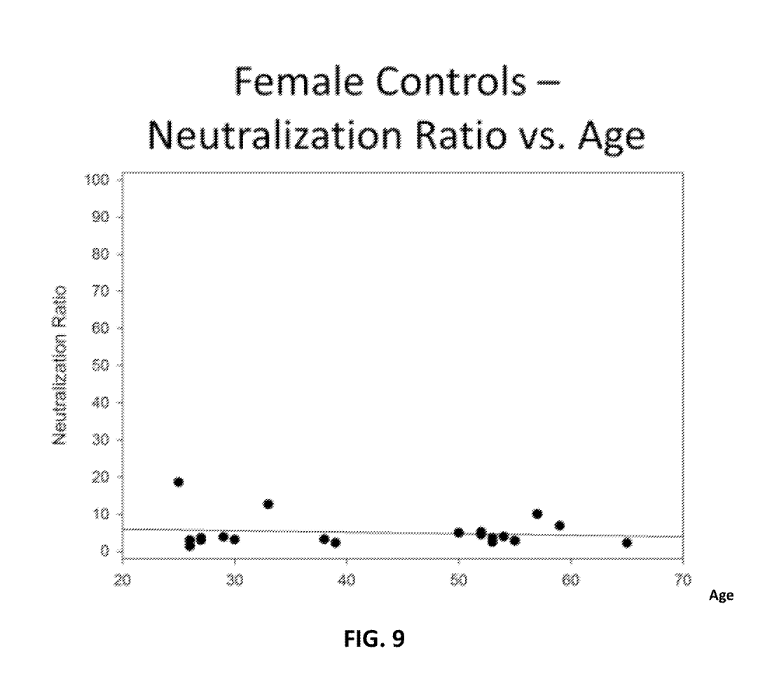

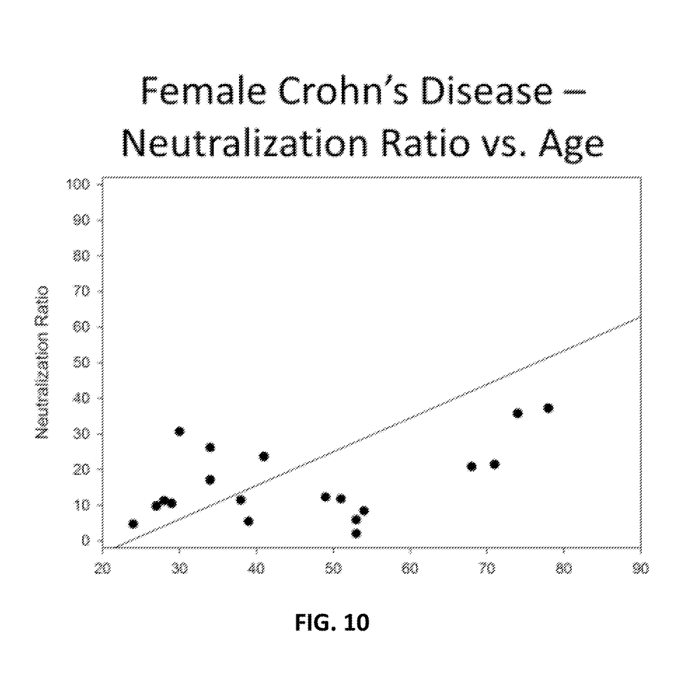

The control female “Endotoxin Neutralization Ratio” samples had a pattern similar to the control males. Sixteen of the samples were clustered in an area between 1.36 to 5.17. There were an additional 4 samples spread out between 6.88 to 18.60. The female CD patients gave higher values over a larger range. There were two patients that gave values like the average control (2-5) and two more patients that were slightly higher (in the 5-6 range). Next was a cluster of 7 patients between 8 to 13. Higher than this there were two populations, one with 5 patients between 17 to 27 and another with 3 patients between 30-40. Lastly, there was one patient with a ratio of 153.17. As with the males, medication may play a role in ratio severity, however, in the females, age was also a factor. The 3 oldest patients had the highest ratios and the youngest 4 patients were among the lowest 8. A trend line comparing Endotoxin Neutralization Ratio vs. patient age in both female controls and CD is shown (FIGS. 9 and 10). The slope of the trend line in the controls is almost horizontal with a value of −0.0427. In the CD patients the trend increases with age with a slope of 0.9474. In addition to age, medication may be a factor. Three of the highest 4 ratios were not prescribed an immunomodulator, antibiotic or anti-inflammatory. Conversely, 15 of the 17 lowest ratios were prescribed at least one of these drug types.

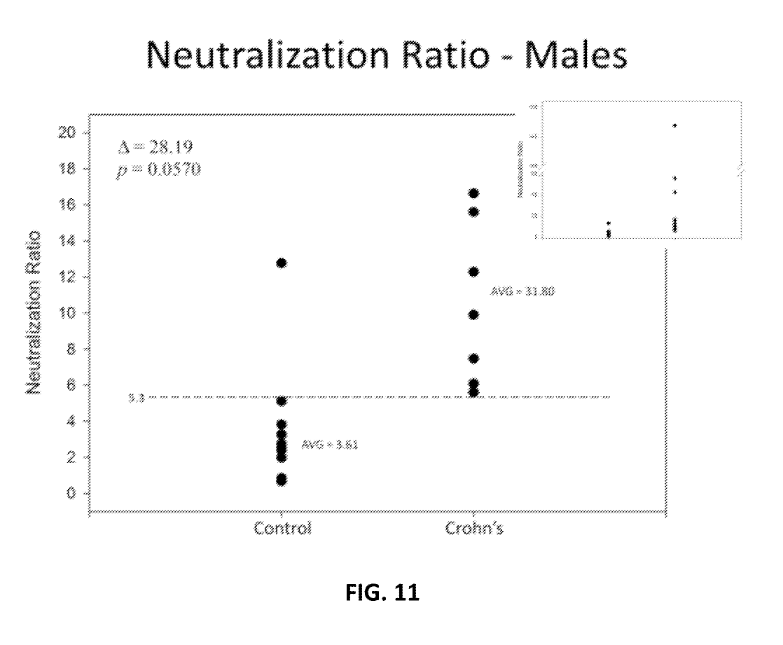

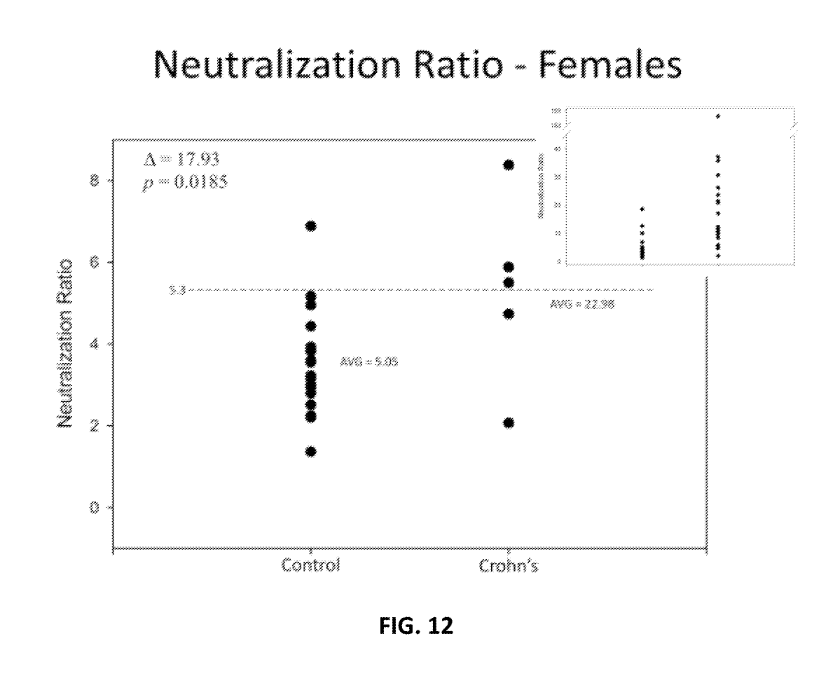

In addition to indicating disease severity, the Endotoxin Neutralization Ratio can demarcate control from disease patients with high accuracy. If a line is included on each graph at a value of 5.3, 9/10 male controls, 10/10 male CD, 16/20 female controls and 18/20 female CD can be distinguished (FIGS. 11 and 12). This represents an accuracy of 95% in males, 85% in females and 88% in all patients.

### Ulcerative Colitis

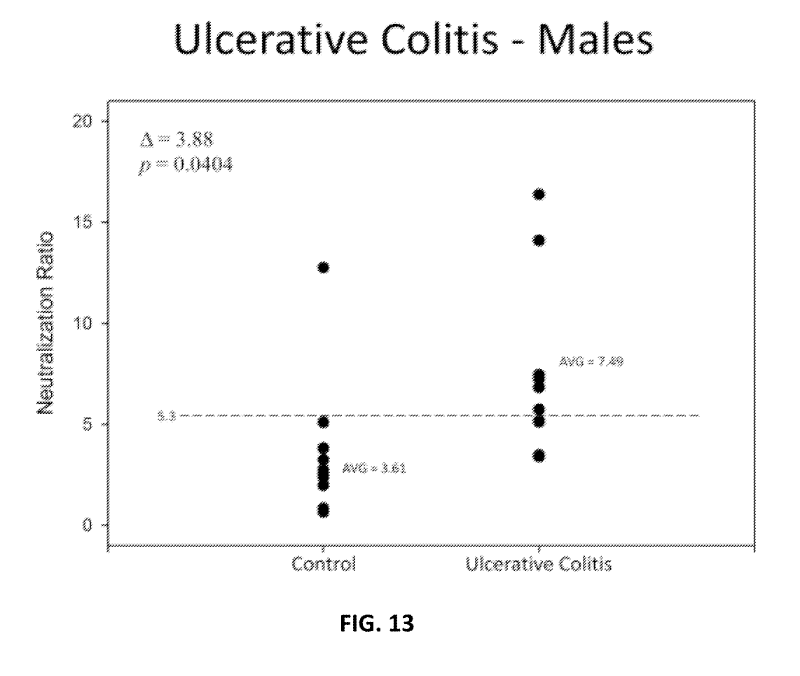

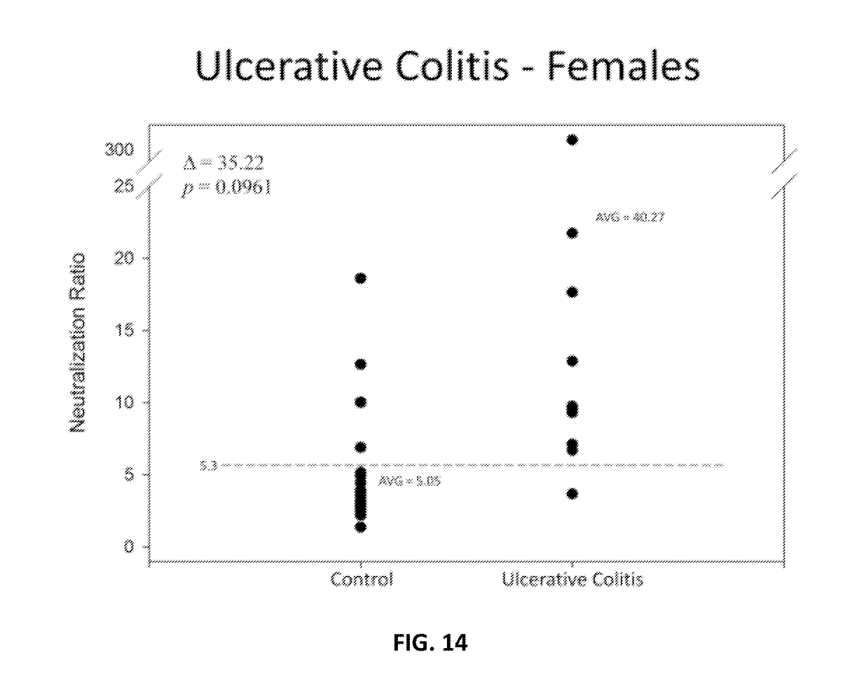

A similar study was conducted with Ulcerative Colitis (UC) patients. Data suggest that the Endotoxin Neutralization Ratio can also be useful in predicting intestinal disease state in these samples. Summarily, the UC data is not as severe as in the CD patients and the ratios not as diverse. In males, the average ratio increases from 3.62 in controls to 7.49 in UC. The line at 5.3 correctly distinguishes 9/10 controls and 6/10 UC samples for an accuracy of 75%. In females the average ratio increases from 5.05 in controls to 40.27 in UC. The line at 5.3 correctly distinguishes 16/20 controls and 9/10 UC samples for an accuracy of 83% (FIGS. 13 and 14).

This study demonstrates the utility of using endotoxin neutralization as a biomarker in intestinal disorders. Differences were observed in Total Endotoxin Neutralization, Enzymatic Endotoxin Neutralization, Protein Endotoxin Neutralization and Undetectable Endotoxin between sexes and/or between control and disease groups. These individual results were used to assign an Endotoxin Neutralization Ratio to each patient sample which was useful in distinguishing control from disease patients as well as distinguishing age, disease severity and medication history in some samples. Table 1 below summarizes the results of using the Endotoxin Neutralization Ratio in distinguishing control from disease states.

TABLE 1

| | | # | # Samples | |
|---|---|---|---|---|
| | | Samples | Correctly | % |
| Control/Disease | Sex | Tested | Predicted | Accuracy |
| Control | Male | 10 | 9 | 90% |
| Control | Female | 20 | 16 | 80% |
| Crohn's Disease | Male | 10 | 10 | 100% |
| Crohn's Disease | Female | 20 | 18 | 90% |
| Ulcerative Colitis | Male | 10 | 6 | 60% |
| Ulcerative Colitis | Female | 10 | 9 | 90% |
| All Samples | Male | 30 | 25 | 83% |
| All Samples | Female | 50 | 43 | 86% |
| All Samples | Both | 80 | 68 | 85% |

1. A method of determining the effectiveness of a therapeutic agent for treating intestinal wall damage comprising:
a) administering a therapeutic agent to the subject;
b) adding exogenous endotoxin to a first biological sample from the subject after treatment with the therapeutic agent;
c) detecting exogenous endotoxin in the sample from step b);
d) determining the amount of undetected exogenous endotoxin in the first sample by subtracting the detected exogenous endotoxin from step c) from the total exogenous endotoxin added in step b); and
e) calculating the percentage of total endotoxin neutralization in the first sample, wherein the percentage of total endotoxin neutralization is the percentage of undetected exogenous endotoxin of the total exogenous endotoxin, and wherein a decrease in total endotoxin neutralization in the first sample as compared to control indicates that the therapeutic agent is effective for treating intestinal wall damage.

2. The method of claim 1 further comprising:
f) heating a second biological sample from the subject after treatment with the therapeutic agent;
g) adding exogenous endotoxin to the second sample of step f);
h) detecting exogenous endotoxin in the second sample from step g);
i) determining the amount of undetected exogenous endotoxin by subtracting the detected exogenous endotoxin from step h) from the amount of added exogenous endotoxin from step g); and
j) calculating the percentage of enzymatic endotoxin neutralization utilizing the following equation:
((amount of undetected exogenous endotoxin in the second sample from step h−amount of undetected exogenous endotoxin in the first sample)/amount of total exogenous endotoxin added in step g)×100;
wherein an increase in the percentage of enzymatic endotoxin neutralization in the second biological sample as compared to control indicates that the therapeutic agent is effective for treating intestinal wall damage.

3. A method of determining the effectiveness of a therapeutic agent for treating intestinal wall damage comprising:
a) administering a therapeutic agent to the subject;
b) heating a first biological sample from the subject after treatment with the therapeutic agent;
c) adding a selected amount of exogenous endotoxin to the first sample of step b);
d) detecting exogenous endotoxin in the first sample from step c);
e) determining the amount of undetected exogenous endotoxin in the first sample by subtracting the detected exogenous endotoxin from step d) from the total exogenous endotoxin added in step c);
f) acidifying a second biological sample from a subject after treatment with the therapeutic agent;
g) adding the selected amount of exogenous endotoxin to the second sample of step f);
h) detecting exogenous endotoxin in the second sample from step g);
i) determining the amount of undetected exogenous endotoxin in the second sample by subtracting the detected exogenous endotoxin from step h) from the total exogenous endotoxin added in step g); and
j) calculating a percentage of protein endotoxin neutralization, utilizing the following equation:
((amount of undetected exogenous endotoxin the second sample−amount of undetected exogenous endotoxin in the first sample)/selected amount of exogenous endotoxin)×100;
wherein a decrease in the percentage of protein endotoxin neutralization after treatment as compared to a control indicates that the therapeutic agent is effective for treating intestinal wall damage.

4. A method of treating intestinal wall damage in a subject comprising:
a) calculating levels of protein endotoxin neutralization, undetected exogenous endotoxin and enzymatic endotoxin neutralization in a biological sample from the subject; and
b) calculating an endotoxin neutralization ratio using the following formula:
(protein endotoxin neutralization+undetectable exogenous endotoxin)/enzymatic endotoxin neutralization)
a ratio of about 5 or greater indicating that the subject has intestinal wall damage; and
c) administering a therapeutic agent for treating intestinal wall damage to the subject.

5. The method of claim 4, wherein protein endotoxin neutralization is calculated by:
a) heating a first biological sample from the subject;
b) adding a selected amount of exogenous endotoxin to the first sample of step a);
c) detecting exogenous endotoxin in the first sample from step b);
d) determining the percentage of undetected exogenous endotoxin in the first sample using the following equation:
((total exogenous endotoxin added in step b−the detected exogenous endotoxin from step c)/the total exogenous endotoxin added in step b)×100;
e) acidifying a second biological sample from a subject;
f) adding the selected amount of exogenous endotoxin to the second sample of step e);
g) detecting exogenous endotoxin in the second sample from step f);
h) determining the percentage of undetected exogenous endotoxin after acidification using the following equation:
((total exogenous endotoxin added in step f−the detected exogenous endotoxin from step g)/the total exogenous endotoxin added in step f)×100;
and
i) calculating protein endotoxin neutralization, utilizing the following equation:
percentage of undetected exogenous endotoxin in step h)−percentage of undetected exogenous endotoxin in step d).

6. The method of claim 4, wherein undetectable endotoxin is calculated by:
a) acidifying a biological sample from a subject;
b) adding a selected amount of exogenous endotoxin to sample of step a);
c) detecting exogenous endotoxin in the sample from step b); and
d) determining the percentage of undetectable exogenous endotoxin after using the following equation:
((total exogenous endotoxin added in step b−the detected exogenous endotoxin from step c)/the total exogenous endotoxin added in step b)×100;

7. The method of claim 4, wherein enzymatic endotoxin neutralization is calculated by:
a) adding exogenous endotoxin to a first biological sample from the subject;
b) detecting exogenous endotoxin in the first sample from step a);
c) determining the amount of undetected exogenous endotoxin in the first sample by subtracting the detected exogenous endotoxin from step b) from the total exogenous endotoxin added in step a);
d) calculating the percentage of undetected exogenous endotoxin in the first sample using the following equation:
(the amount of undetected exogenous endotoxin in step c/total exogenous endotoxin added in step a)×100;
e) heating a second biological sample from the subject;
f) adding exogenous endotoxin to the second sample of step e);
g) detecting exogenous endotoxin in the second sample from step f);
h) determining the amount of undetected exogenous endotoxin in the second sample by subtracting the detected exogenous endotoxin from step g) from the amount of added exogenous endotoxin from step f);
i) calculating the percentage of undetected exogenous endotoxin in the second sample using the following equation:
(the amount of undetected exogenous endotoxin in step h/total exogenous endotoxin added in step f)×100;
j) calculating the percentage of enzymatic endotoxin neutralization utilizing the following equation:
((percentage of undetected exogenous endotoxin in the second sample−percentage of undetected exogenous endotoxin in the first sample)/amount of total exogenous endotoxin added in step a)×100.

8. The method of claim 5 wherein the acidification step comprises acidification to a pH of about 1 to 4.

9. The method of claim 8 further comprising:
a) contacting the acidified sample with an acidic protease; and
b) increasing the pH of the protease-treated sample to a pH of about 6 to 8.

10. The method of claim 1, wherein the sample is selected from the group consisting of plasma, blood, serum, ascites, pleural fluid, ocular fluid and spinal fluid.

11. The method of claim 10, wherein the plasma is citrated plasma or EDTA collected plasma.

12. The method of claim 10, wherein the acidic protease is a pepsin.

13. The method of claim 2, wherein the biological sample is heated to a temperature of about 55° C. to about 70° C.

14. The method of claim 8, wherein the biological sample is diluted to about 1 part sample to about 10 parts diluent prior to acidification.

15. (canceled)

16. The method of claim 9, wherein the biological sample is diluted to about 1 part sample to about 10 parts diluent prior to acidification of the protease-treated sample.

17. (canceled)

18. (canceled)

19. The method of claim 8, further comprising inactivating the acidic protease.

20. The method of claim 1, further comprising mixing about 9 parts biological sample with about 1 part endotoxin free water prior to step a).

21. The method of claim 2, further comprising mixing about 9 parts biological sample with about 1 part endotoxin free water prior to step a).

22. The method of claim 3, wherein the biological sample is acidified by adding 1 part acidic solution to about 9 parts biological sample.

23. The method of claim 1, wherein 1 part exogenous endotoxin is added to about 9 parts biological sample.

24-27. (canceled)
